(19) United States
(12) Patent Application Publication
(10) Pub. No.: US 2009/0143792 A1
(43) Pub. Date: Jun. 4, 2009

(54) Holding Device for a Membrane, Support for the Holding Device, and Method for Fixing the Membrane to an Eye by Means of the Holding Device

(76) Inventors: Katrin Engelmann, Dresden (DE); Heinz Korber, Dresden (DE); Carsten Werner, Dresden (DE)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/296,679

(22) PCT Filed: Apr. 5, 2007
(86) PCT No.: PCT/DE2007/000681

§ 371 Date: Jan. 26, 2009

(30) Foreign Application Priority Data
Apr. 13, 2006 (DE) 10 2006 019 017.3

(51) Int. Cl. A61F 9/007

(52) U.S. Cl. 606/151

(57) ABSTRACT

The invention relates to a holding device (10) for an amnion membrane (20), wherein the amnion membrane can be mounted on the eye surface without a suture or any other securing means for medical application. For this purpose, the holding device comprises concentrically arranged rings (12, 14), wherein a first ring acting as inner ring (14) is accommodated in a receiving seat (12a) on the inner face of a second ring acting as outer ring (12), such that the two rings have a common frustoconical inner surface (12b), and wherein the amnion membrane (20) in the area of this receiving seat (12a) is clamped peripherally between the two rings (12, 14) and is arranged covering the common opening (18) of the two rings.

The invention relates to a fixture for a membrane in a medical application.

In treatments of ocular surfaces, for example, membranes are placed thereon and are then immobilized on the eye.

It is known to dispose membranes for wound healing on the ocular surface by gluing or suturing. This kind of attachment can lead to problems because the attachment of the membrane takes place as a rule in healthy regions of the ocular surface uninvolved in the required wound healing and in isolated cases can lead to irritations, hemorrhages, the formation of scars or the like in these asymptomatic regions. What is more, blinking can produce forces on the membrane positioned on the ocular surface, which forces can lead to dislodgement or even detachment of the membrane, necessitating a renewed attachment. It is further known to employ a crosslinked membrane made from an amniotic membrane.

Proceeding from this prior art, it is an object of the invention to dispose a membrane on an ocular surface in simple and safe fashion, both the preparatory treatment of the membrane and also the mounting in the eye taking place with as little stressing as possible.

In order to achieve this object, a fixture having the features of Claim 1 is proposed.

The fixture according to the invention—wherein the membrane is disposed on two rings disposed concentrically to one another, a first ring, as inner ring, being accommodated by a receptacle on the inner side of a second ring, as outer ring—offers the possibility of clamping the membrane in the receptacle in a fashion safe against loss, without the necessity of further attachment means between the two rings. Here the membrane is gripped between the two rings at its circumference and freely spans the common opening of the concentrically disposed rings. Upon placement on the human eye, the freely stressed section of the membrane can come into contact, under stress, with the ocular surface. For adaptation to the convexity of the human eye, the common inner side that arises upon the disposition of the inner ring in the receptacle of the outer ring is fashioned as a truncated cone narrowing toward the membrane. The inner ring has its inner side flush with the inner side of the outer ring in which it is accommodated, so that a common inner surface of the fixture in the shape of a truncated cone is formed. In this way it is possible to place membranes on the ocular surface in a fashion free of seams and thus gentle to the tissue and accurately fitting.

It is advantageous here if the inner ring is substantially rigid while the outer ring is elastic. The stability of the fixture comprising the two mutually disposed rings is thus based on the inner ring, while the outer ring, which comes into contact with the sensitive parts of the eye in the medical application, is fashioned as elastic and thus softer in order to avoid irritation or even injury to the already damaged eye. The elastic fashioning of the outer ring also makes it possible to position the inner ring in the receptacle of the outer ring in simple fashion by drawing the elastic outer ring over the rigid inner ring. Because the membrane is also non-positively mounted between the two rings given this way of disposing the inner ring, the risk of damaging the membrane is also minimized by the elastically soft fashioning of the one ring.

It is further appropriate here to cover the outside and top of the rigid inner ring substantially completely with the outer ring in order to protect the eye. In this embodiment the receptacle in the outer ring has an opening only on the inner side of the ring, which opening is closed by the inner side of the inner ring lying flush with the inner side of the outer ring, so that there are no edges or projections.

To this end, in an appropriate development of the invention, the outer ring exhibits a cross section fashioned substantially as a triangle having a wedge-shaped recess for the inner ring. Here one of the sides of the triangle, beginning in the region of one of the vertices of the triangle, is partly formed by one side of the wedge-shaped recess. This means that in the assembled position of both rings a part of this side is formed by the inner side of the inner ring. The tip belonging to the angle opposite this side of the triangle is fashioned as a radiused edge, because this edge forms the part of the fixture that is farthest away from the ocular surface upon insertion into the eye and from which the greatest loading for the eye originates. It is therefore advantageous to avoid tips and edges in this region.

It has further proved desirable to fashion the outer ring with its lower section projecting beyond the inner ring and tapering acutely. In this region the fixture when inserted into the eye extends particularly deeply into the ocular cavity, the distance between the conjunctiva and the eyeball growing smaller. In order to avoid irritations, the thickness of the outer ring decreases because of the acute taper while the bearing area is maximal. The resulting sharp-edged termination offers no area of attack in case of movements of the fixture on the eye.

At the same time, the inner ring advantageously exhibits a wedge-shaped cross section whose tip points toward the membrane framed between the two rings, which rests on the top of the inner ring. In the region where the membrane is introduced between the two rings, each of these is fashioned acutely converging toward the other so as to form a slot. In this way the force expended when drawing the elastic outer ring over the rigid inner ring is made small, as is the change in direction necessary for insertion of the membrane between the two rings.

The opening angle of the common truncated-cone-shaped inner surface of the two cooperating rings appropriately lies in the range of the convexity of a human eyeball and is preferably equal to 97°, because then the whole fixture bears on the eye in the most accurately fitting fashion, and the membrane can be brought to bear on the ocular surface over its free area in the region of the common ring opening. Because the bearing of the inner side of both rings makes the most accurate possible fit, the eyeball protrudes partway through the common concentric ring opening and is thus in contact with the membrane in order that the healing action of said membrane can be deployed in particular for the centrally located cornea.

An amniotic membrane derived from the human placenta exhibits particular suitability for this medical application on the eye.

Advantageous embodiments of the invention consist in charging the membranes employed with growth factors.

For fashioning the rings, a rigid thermoplastic material for the inner ring and an elastic silicone for the outer ring have proved particularly advantageous. Both materials make possible a simple fabrication of the fixture, and their use is unobjectionable in terms of health.

The object underlying the invention is further achieved with a support having the features of Claim 12. The support according to the invention for the above-cited fixture exhibits a support body. The support body is a cylinder having a circular cross section and an outwardly convex end face. The outwardly directed convexity of the end face here corresponds approximately to the contour and convexity of a human eyeball in the region of the cornea.

The diameter of the end face at its base is substantially identical to the outside diameter of the inner ring of the fixture.

With this fashioning of the support, the fixture with the membrane can be disposed on the support in such fashion that the membrane can be brought to bear on the support body, the support body exhibiting substantially the contour of an ocular surface. In this way the membrane is prepositioned and can be prepared and/or given preservative treatment while in the application position. In this way, changes in shape that can lead to a diminished adaptation to the ocular surface are markedly reduced. While on the support, the ready-to-use construct of the fixture stressing the membrane can be stored ready for use up until the operation and then, during the operation, placed in use in simple fashion. The support body itself can be mounted on a base plate.

In an appropriate development for further adapting the support to the shape of the fixture, the marginal regions of the end face are fashioned in the shape of a truncated cone, the opening angle of the truncated cone appropriately being equal to the opening angle of the truncated cone of the fixture.

In an alternative embodiment, the support body of the support exhibits, in the region of the base of the end face, a circumferential groove having at least the depth of the radial extent of the inner ring of the fixture. With this fashioning the fixture rests on the support body substantially only with the stressed membrane. Said membrane is subject to a permanent tensile stress solely because of the gravity force acting on both rings, which tensile stress prevents the membrane from slipping, folding or the like and thus makes possible a substantially smooth bearing on the ocular surface when the fixture is used in the human eye.

In order to achieve the object underlying the invention, there is further proposed a method having the features of Claim 15. The supporting innovation here is to position the membrane on the eye being treated using the fixture according to the invention, the fixture in the eye being held on the cornea and the conjunctiva solely by the lid of the eye and the membrane now being brought to rest smoothly on the ocular surface. No additional loading of the eye due to a connection of the membrane to the eye is necessary. Nor is there any further loading of the membrane by such a connection, which further loading might lead to undesired changes in the membrane or damage thereto. The membrane is not further altered in the course of the eye operation but is inserted into the eye with the mounting in which it is presented to the operating surgeon. Suturing, gluing or similar inversive connection to parts of the eye is not necessary with the method according to the invention.

The fixture with the gripped membrane is advantageously already available and ready for use before the operation. The eye operation proper can then be limited to positioning the fixture with the membrane and to the actions necessary for this purpose. There is no further need for alterations to the fixture or the membrane; instead, the fixture with the membrane is inserted into the eye as a complete unit.

It is further appropriate to prepare the membrane in the fixture before insertion into the eye. The finally prepared membrane is then not subject to any further loadings due to insertion or immobilization in a mounting.

For the same reason it is advisable to apply preservative treatment to the membrane in the fixture before insertion into the eye. With a preservative treatment, for example by deep-freezing the membrane in a nutrient medium, the prior gripping makes it possible to avoid creases in the membrane during the preservative treatment, which creases can hardly be completely eliminated again when the membrane is placed on the eye.

One membrane particularly suitable for this medical application with the fixture according to the invention is an amniotic membrane.

Further advantages and features of the invention can be inferred from the following description of the exemplary embodiments and from the individual claims.

In the Drawings:

FIG. 1 depicts in perspective view a fixture for a membrane;

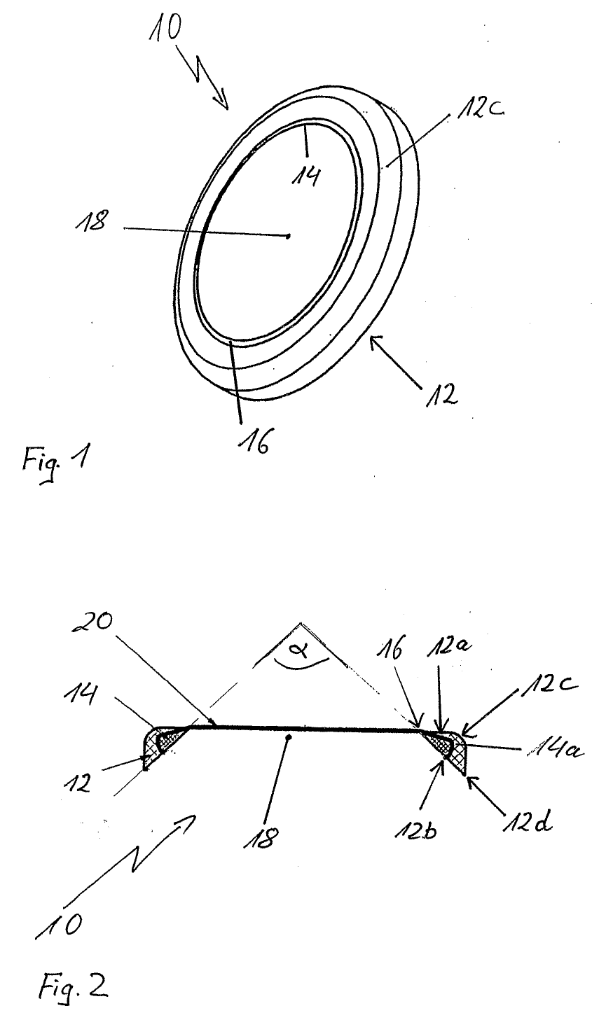

FIG. 2 depicts in sectional view the fixture of FIG. 1 having a framed membrane;

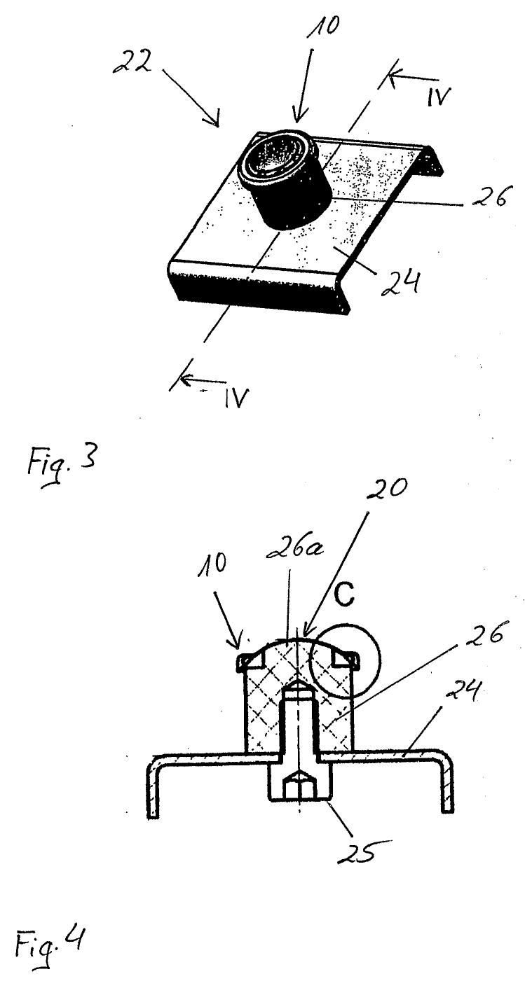

FIG. 3 depicts in perspective view a support for the fixture;

FIG. 4 depicts, in cross-sectional view along line IV-IV, the support according to FIG. 3;

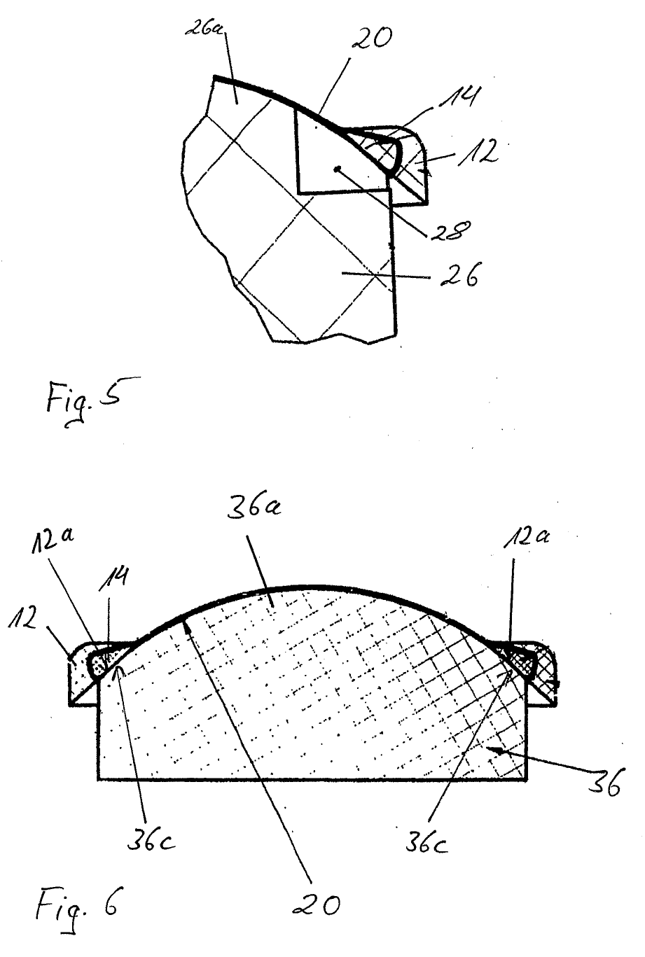

FIG. 5 depicts the detail “C” according to FIG. 4; and

FIG. 6 depicts in cross-sectional view a support for the fixture in an alternative embodiment.

FIG. 1 depicts a fixture 10 in which a membrane can be accommodated and, with the fixture, brought into contact with the ocular surface of a human eye for a medical treatment.

The placement of an amniotic membrane derived from the placenta after a caesarean delivery makes it possible at least to alleviate problems with wound healing, in particular of the corneal epithelium, after injury or other damage to the cornea.

In order to dispose a membrane, not depicted in FIG. 1, on the fixture for this purpose, that is, so that it can be gripped, the fixture comprises an outer ring 12 that is so elastic that it can be drawn over a rigid inner ring 14, of which only a tip can be seen in FIG. 1. In the assembled position, inner ring 14 forms in the region of its tip a flexible slot 16 with outer ring 12, in which slot a membrane can be accommodated and immobilized. The elastic fashioning of outer ring 12 has the result that slot 16 is flexible and the membrane can be mounted under stress.

Inner ring 14 and outer ring 12 are disposed concentrically to one another and exhibit a common concentric opening 18, which can then be spanned by a membrane. Here both rings are fashioned as rotationally symmetrical bodies.

FIG. 2 depicts fixture 10 as it is placed in the eye, which is not depicted. The fixture exhibits as membrane an amniotic membrane 20, which is laid on rigid inner ring 14 made of PEEK and fixed by elastic outer ring 12 made from silicone. Rigid inner ring 14 is fashioned with a wedge-shaped cross section, the tip of the wedge pointing into the region of slot 16 on the top of the fixture. Elastic outer ring 12 is fashioned with a receptacle 12a and, by virtue of its elasticity, can be drawn over inner ring 14 in such fashion that inner ring 14 is finally accommodated in this receptacle 12a flush with inner surface 12b of outer ring 12. With the exception of the part of inner surface 12b formed by inner ring 14 and of slot 16 between the two rings, outer ring 12 completely encloses inner ring 14.

Because of its elastic fashioning, the outer ring encloses the inner ring under stress, so that the rigid inner ring can be released from its connection with outer ring 12 only by the exertion of appropriate force, receptacle 12a being fashioned with an undercut in relation to removal of the inner ring from the receptacle, and edge 14a of inner ring 14 intruding into receptacle 12a being radiused in order to reduce the risk of damage to outer ring 12 in the region of its receptacle 12a by the rigid inner ring.

Now, without further mechanical means of attachment, circular amniotic membrane 20 is clamped at its circumference between the top of inner ring 14 and outer ring 12 enclosing said inner ring and extends over entire common opening 18 of the two rings, the amniotic membrane being attached to the rings under a certain prestress. Amniotic membrane 20 is freely stressed in the region of concentric common opening 18 of rings 12, 14 and, for the purpose of medical applications, can be brought to bear on the surface of a human eye.

Outer ring 12 likewise exhibits a substantially triangular cross section, the tip of this triangle, which is in itself a right triangle, being radiused at its vertex opposite the inner side 12b forming the hypotenuse of the triangle, since circumferential edge 12c of outer ring 12, formed by this tip, is positioned directly under the eyelid and held thereby when fixture 10 is positioned in the eye. With this cross section, inner surface 12b of rotationally symmetric outer ring 12 forms a truncated cone open at the top and bottom, which narrows upwardly and exhibits an opening angle α of approximately 97° adapted to the convexity of a human eye.

On the open bottom of the truncated cone, outer ring 12 exhibits an acutely converging edge 12d with which fixture 10 is supported on the eye. Circularly circumferential edge 12d of elastic outer ring 12 made of silicone has a diameter of 19.8 mm versus a diameter of 17.4 mm in the region of the outer edge of receptacle 12a on inner surface 12b, so that fixture 10 can be brought to bear only with the softer outer ring in the marginal region of the cornea of the eye, and the surface of the outwardly convex eye can be brought to bear with the underside of amniotic membrane 20 inside circumferential edge 12d of fixture 10. In the region of the upper termination of fixture 10, in which upper termination the amniotic membrane is stressed, opening 18 has a diameter of 14.2 mm.

In the position of the fixture in the eye, said fixture comes into contact with the ocular surface, edge 12d being slid under the lid. The movable lid covering the ocular surface holds fixture 10 by continuously covering the region of radiused edge 12c of the fixture, the entire fixture with the upper side of amniotic membrane 20 being covered by the lid during a blink. The maximum thickness of fixture 10 relative to the ocular surface is 1.5 mm, which thickness raises radiused edge 12c above inner surface 12b of outer ring 12.

In FIG. 3, fixture 10 with amniotic membrane 20 is disposed on a support comprising a base plate 24 and a support body 26. Fixture 10 is set on support body 26.

As can be seen in particular from FIG. 4, support body 26 is attached to base plate 24 with a screw 25 from its bottom, the base plate being curved on two opposite sides so that the base plate forms a stable base for support body 26.

Support body 26 is a body fashioned as a circular cylinder with one flat end face resting flush against base plate 24 and is fashioned as outwardly convex at opposite end face 26a. The convexity of support body 26 here corresponds to a spherical cap having a spherical radius of 12 mm, which arches above the circular cylinder of support body 26 having a radius of 8.7 mm. The diameter of the circular cylinder of support body 26 is so chosen that the fixture projects radially outwardly relative to the support body starting at the transition at inner surface 12b from rigid inner ring 14 to soft elastic outer ring 12.

Here fixture 10 is set on convex end face 26a, amniotic membrane 20 resting with its bottom side on the convex end face, whose contour substantially corresponds to that of a human eye.

As illustrated in FIG. 5, support body 26 exhibits a partially or completely circumferential groove 28 of substantially triangular cross section at the base of the spherical cap forming end face 26a, which groove extends radially into support body 26 in the transition region between the cylindrical base body and the spherical cap of support body 26. With this fashioning, fixture 10 rests with rings 12, 14 only on the marginal region of support body 26, amniotic membrane 20 resting on the spherical cap of end face 26a and in this way standing under a smoothing tensile stress.

As depicted in FIG. 6, in an alternative embodiment of an end face 36a of a support body 36—otherwise identical but fashioned without a base plate—of a support 22, the spherical cap forming the convex end face is made to transition at its base, that is, outwardly, into a truncated cone shape 36c adapted to the inner surface 12b of rings 12, 14, which can be brought to rest at this point, so that rings 12, 14 having common lateral inner surface 12b can be brought to bear flatly on section 36c of convex end face 36a.

Even before the medical application, amniotic membrane 20, when disposed in fixture 10 wherein it is placed in the eye for the purpose of said medical application, can be stored in a nutrient solution, also deep-frozen, and/or made accessible for a biochemical or tissue-altering manipulation, for example for crosslinking with proteins or for the placement of growth factors.

In these procedures before the operation proper, amniotic membrane 10, in a disposition of fixture 10 on support 22, is also positioned in well-defined fashion and protected against damage. As a result of the applied tensile stress, convex end face 26a, 36a of support body 26, 36 protects the amniotic membrane against folding, so that the direct contact with the cornea, as free of folding as possible, necessary for the wound-healing action is improved.

After storage and preparation, the amniotic membrane is furnished to the operating surgeon ready for use in fixture 10 and, as appropriate, in combination with support 22, and said surgeon can place the fixture in the eye without further removal or attachment steps.

Because amniotic membrane 20 is already stored in fixture 10, alterations to the membrane are markedly reduced and the amniotic membranes exhibit a high optical quality.

The medical application of the teaching according to the invention is preferably to be seen in the human realm but is not limited thereto.

1. A fixture for a membrane for a medical application, having two rings disposed concentrically to one another, a first ring, as inner ring, being accommodated by a receptacle on the inner side of a second ring, as outer ring, in such fashion that the two rings exhibit a common inner surface in the shape of a truncated cone, and the membrane being circumferentially gripped between the two rings in the region of this receptacle and disposed so as to cover the common opening of the two rings.

2. The fixture of claim 1, wherein the inner ring is substantially rigid and the outer ring is elastic.

3. The fixture of claim 1, wherein the outer ring is disposed so as to cover the inner ring on its outer and upper side.

4. The fixture of claim 1, wherein the outer ring exhibits a base surface fashioned substantially as a triangle having a wedge-shaped recess forming the receptacle, having one side of the triangle partly formed by one side of the wedge-shaped recess proceeding from one of its vertices, and having one tip forming a radiused edge at the angle opposite this side.

5. The fixture of claim 1, wherein the outer ring is fashioned with its lower section projecting beyond the inner ring and having an acutely tapering edge.

6. The fixture of claim 1, wherein the inner ring exhibits a wedge-shaped cross section.

7. The fixture of claim 1, wherein the common, truncated-cone-shaped inner surface of the two cooperating rings exhibits an opening angle α in the range of the convexity of a human eyeball.

8. The fixture of claim 7, wherein the opening angle α is approximately 97°.

9. The fixture of claim 1, wherein the membrane is an amniotic membrane (20).

10. The fixture of claim 1, wherein the inner ring is made of a thermoplastic.

11. The fixture of claim 1, wherein the outer ring is made of silicone.

12. A support for a fixture fashioned according to claim 1, having a support body, the support body being a cylinder having a circular cross section and an outwardly convex end face, the convexity of the end face approximately corresponding to the convexity of a human eyeball in the region of the cornea, and the diameter of the end face at its base being substantially identical to the outside diameter of the inner ring of the fixture.

13. The support of claim 12, wherein the circumferential marginal regions of the end face are fashioned in the shape of a truncated cone at the base of said end face.

14. The support of claim 12, having a circumferential groove in the region of the base of the end face, the groove exhibiting at least the radial depth of the radial extent of the inner ring of the fixture.

15. A method for immobilizing a membrane on the surface of an eye for the purpose of a medical application, having the membrane gripped in a fixture according to claim 1, having the insertion of the fixture with the membrane in the eye, the fixture being held in the eye solely by the lid of said eye and the membrane thus being brought to rest smoothly on the ocular surface.

16. The method of claim 15, wherein the fixture with the gripped membrane is furnished ready for use before the operation.

17. The method of claim 15, wherein the membrane is prepared in the fixture before insertion into the eye.

18. The method of claim 15, wherein the membrane is given preservative treatment in the fixture before insertion into the eye.

19. The method of one of claim 15, wherein an amniotic membrane is employed as the membrane.
